(19) United States
(12) Patent Application Publication
(10) Pub. No.: US 2026/0215664 A1
(43) Pub. Date: Jul. 30, 2026

(54) MEDICAL SYSTEMS AND DEVICES HAVING AN ARTICULABLE EXTERNAL CHANNEL AND RELATED METHODS OF USE

(71) Applicant: Boston Scientific Scimed, Inc., Maple Grove, MN (US)

(72) Inventors: Ryan Davis GILKEY, Austin, TX (US); Alexander Ross COHEN, Austin, TX (US); Timothy Noah FOSTER-FRAU, Austin, TX (US)

(73) Assignee: Boston Scientific Scimed, Inc., Maple Grove, MN (US)

(21) Appl. No.: 19/457,298

(22) Filed: Jan. 23, 2026

Related U.S. Application Data
(60) Provisional application No. 63/750,960, filed on Jan. 29, 2025.

(51) Int. Cl. A61B 1/018; A61B 1/00; A61B 1/005

(52) U.S. Cl. CPC A61B 1/018; A61B 1/00135; A61B 1/0053; A61B 1/0057

(57) ABSTRACT

A medical system includes a first medical device and a second medical device coupled to at least a portion of the first medical device. The first medical device includes a handle and a shaft extending distally from the handle. The second medical device includes a distal portion having a plurality of ribs and a channel. The channel extends through the plurality of ribs. The second medical device includes a plurality of cables coupled to the plurality of ribs. Each cable of the plurality of cables is configured to move the distal portion of the second medical device from a first configuration, in which the distal portion of the second medical device is aligned with a longitudinal axis of the shaft, to a second configuration, in which the distal portion of the second medical device is at a non-zero angle relative to the longitudinal axis of the shaft.

### CROSS-REFERENCE TO RELATED APPLICATION(S

This application claims the benefit of priority to U.S. Provisional Application No. 63/750,960, filed on January 29, 2025, which is incorporated by reference herein in its entirety.

### TECHNICAL FIELD

Various aspects of this disclosure relate generally to articulable medical systems, devices, and related methods of use. In particular, some aspects of this disclosure relate to medical systems and devices having an articulable external channel.

### BACKGROUND

Endoscopic procedures may require an operator to apply traction to tissue and/or perform complex tissue manipulations using multiple medical instruments. For example, Endoscopic Submucosal Dissection (ESD) utilizes medical instruments to remove cancerous or other lesions from the gastrointestinal (GI) tract. During ESD, an operator may need to apply traction to tissue using a medical instrument (e.g., a grasper), which can help the operator visualize separation of the GI tract mucosa layer (and lesion) from the GI tract muscle layer. Additionally, the operator may need to remove the lesion using another medical instrument (e.g., an electrosurgical knife). The medical instruments may be passed through a channel of an endoscope in order to remove the lesion. Movement and/or maneuverability of the medical instruments may be limited by movement of the endoscope.

### SUMMARY

Each of the aspects disclosed herein may include one or more aspects of the features described in connection with any of the other disclosed aspects.

In an aspect, a medical system may include a first medical device and a second medical device coupled to at least a portion of the first medical device. The first medical device may include a handle and a shaft extending distally from the handle. The second medical device may include a distal portion having a plurality of ribs and a channel. The channel may extend through the plurality of ribs. The second medical device may also include a plurality of cables coupled to the plurality of ribs. Each cable of the plurality of cables may be configured to move the distal portion of the second medical device from a first configuration to a second configuration. In the first configuration, the distal portion of the second medical device may be aligned with a longitudinal axis of the shaft of the first medical device. In the second configuration, the distal portion of the second medical device may be at a non-zero angle relative to the longitudinal axis of the shaft of the first medical device.

Any of the devices, systems, or methods disclosed herein may include any of the following features, alone or in any combination. A distal end of each of the plurality of cables may be fixed to a distalmost rib of the plurality of ribs. Each of the plurality of ribs may include two openings that extend longitudinally through portions of each of the plurality of ribs. The two openings may be configured to receive the plurality of cables. Each of the plurality of ribs may include a central opening that partially defines the channel. Each of the plurality of ribs may contact the shaft of the first medical device in the first configuration. One or more distal ribs of the plurality of ribs may be separated from the shaft of the first medical device in the second configuration. Each of the plurality of ribs may include a curved surface complementary to an outermost surface of the shaft of the first medical device. A section of the distal portion of the second medical device may be coupled to the shaft of the first medical device via an attachment device. The medical system may include a return mechanism configured to move the distal portion of the second medical device towards the shaft of the first medical device. The return mechanism may include a thread. The thread may wrap around the shaft of the first medical device. The channel of the second medical device may be configured to receive a medical instrument. When the medical instrument is received within the channel of the second medical device, movement of the distal portion of the second medical device may correspondingly move the medical instrument. The plurality of cables may include a first cable and a second cable. The first cable may extend through a portion of each of the plurality of ribs proximate a first lateral end of each of the plurality of ribs. The second cable may extend through a portion of each of the plurality of ribs proximate a second lateral end of each of the plurality of ribs. The second medical device may include a handle having at least one actuator coupled to the plurality of cables. The at least one actuator may be configured to move the plurality of cables proximally or distally to move the distal portion of the second medical device between the first configuration and the second configuration. The at least one actuator may include a slider, a joystick, or a knob. The medical system may include a sheath defining a lumen. The lumen may be configured to receive at least the distal portion of the second medical device and the shaft of the first medical device.

In another aspect, a medical system may include a first medical device having a handle and a shaft extending distally from the handle. The shaft may include a longitudinal axis, an articulation section, and a distal tip. The system may also include a second medical device extending distally alongside at least a portion of the shaft of the first medical device. The second medical device may include a distal portion having a channel, a first cable, and a second cable. The first cable and the second cable may be configured to move proximally or distally to move the channel between a first configuration and a second configuration. In the first configuration, the channel of the second medical device may be aligned with the longitudinal axis of the shaft of the first medical device. In the second configuration, the channel of the second medical device may be at a non-zero angle relative to the longitudinal axis of the shaft of the first medical device.

Any of the devices, systems, and methods disclosed herein may include any of the following features, alone or in any combination. The distal portion may include a plurality of ribs. The channel may extend through the plurality of ribs. A section of the distal portion may be fixed to the shaft of the first medical device at a position on the shaft proximal to the articulation section.

A further aspect relates to a method of treating a target site. The method may include delivering a medical system to the target site, where the medical system may include a first medical device and a second medical device coupled to at least a portion of the first medical device. The method may include articulating a distal portion of the first medical device to a first position, advancing a first medical instrument through a working channel of the first medical device, articulating a distal portion of the second medical device to a second position independent of the distal portion of the first medical device, and advancing a second medical instrument through a channel of the second medical device. The distal portion of the second medical device may include a plurality of ribs. The channel may extend through the plurality of ribs.

### BRIEF DESCRIPTION OF FIGURES

The accompanying drawings, which are incorporated in and constitute a part of this application, illustrate exemplary aspects of this disclosure and together with the description, serve to explain the principles of this disclosure.

FIG. 1A illustrates an exemplary medical system including an exemplary first medical device coupled to an exemplary second medical device, according to aspects of this disclosure.

FIG. 1B illustrates a top view of a distal portion of the medical system of FIG. 1A in a first configuration, according to aspects of this disclosure.

FIG. 1C illustrates a side view of the distal portion of the medical system of FIG. 1A in a second configuration, according to aspects of this disclosure.

FIG. 1D illustrates a top view of the distal portion of the medical system of FIG. 1A in a third configuration, according to aspects of this disclosure.

FIG. 1E illustrates a top view of the distal portion of the medical system of FIG. 1A in a fourth configuration, according to aspects of this disclosure.

FIG. 2 illustrates a schematic view of an exemplary handle of the second medical device of FIG. 1A, according to aspects of this disclosure.

FIG. 3 illustrates another exemplary handle of the second medical device of FIG. 1A, according to aspects of this disclosure.

FIG. 4 illustrates an exemplary usage of the medical system of FIG. 1A, according to aspects of this disclosure.

FIG. 5 illustrates a side view of a distal portion of another exemplary medical system, according to aspects of this disclosure.

### DETAILED DESCRIPTION

Particular aspects of this disclosure are described in greater detail below. The terms and definitions provided herein control, if in conflict with terms and/or definitions incorporated by reference. Wherever possible, the same or similar reference numbers will be used through the drawings to refer to the same or like parts.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body. Proximal and distal directions are labeled with arrows marked “P” and “D,” respectively, throughout various figures.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Relative terms such as “about,” “substantially,” and “approximately,” etc., are used to indicate a possible variation of ±10% of the stated numeric value or range.

Although endoscopes are referenced herein for illustration purposes, it will be appreciated that this disclosure encompasses any suitable medical device configured to allow an operator to access and view internal body anatomy of a subject (e.g., patient) and/or to deliver medical instruments, such as, for example, biopsy forceps, graspers, baskets, snares, probes, scissors, retrieval devices, lasers, and other tools, into the subject’s body. The medical devices herein may be inserted into a variety of body lumens and/or cavities, such as, for example, the urinary tract or gastrointestinal tract. It will be appreciated that, unless otherwise specified, bronchoscopes, duodenoscopes, ureteroscopes, gastroscopes, endoscopic ultrasonography (“EUS”) scopes, colonoscopes, laparoscopes, cystoscopes, aspiration scopes, sheaths, catheters, or any other suitable delivery device or medical device may be used in connection with the features described herein.

Features of the medical systems and devices herein may improve maneuverability of medical instruments, tissue manipulation, and/or tissue traction during a medical procedure, among other aspects. According to some aspects of this disclosure, the medical systems may include a first medical device (e.g., an endoscope or other suitable insertion device) and a second medical device (e.g., an accessory device) coupled to the first medical device. The second medical device may include a channel extending alongside a distal portion of the first medical device. In some aspects, the second medical device may be configured to receive a medical instrument. The channel of the second medical device may be articulated (e.g., bent) independent of the distal portion of the first medical device. For example, during a medical procedure, the channel of the second medical device may be coupled to the distal portion of the first medical device during delivery to a target site. Once at or proximate the target site, the channel of the second medical device may be articulated to at least partially decouple the channel of the second medical device from the distal portion of the first medical device. The articulable channel of the second medical device may allow for medical instruments to approach target sites from different directions or angles of approach than the first medical device, among other aspects. According to some aspects of this disclosure, the medical system may include a medical instrument having an articulable distal portion.

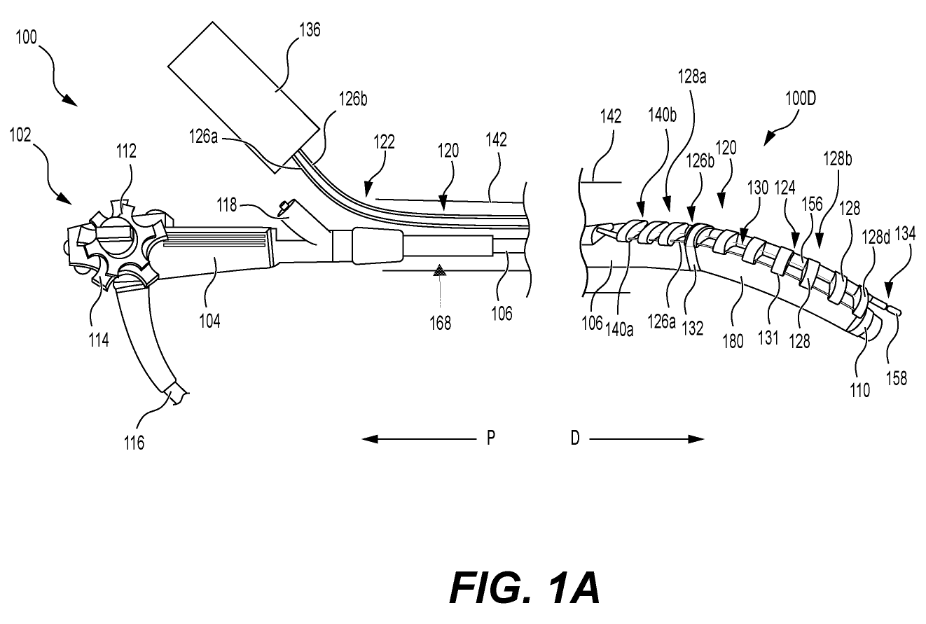

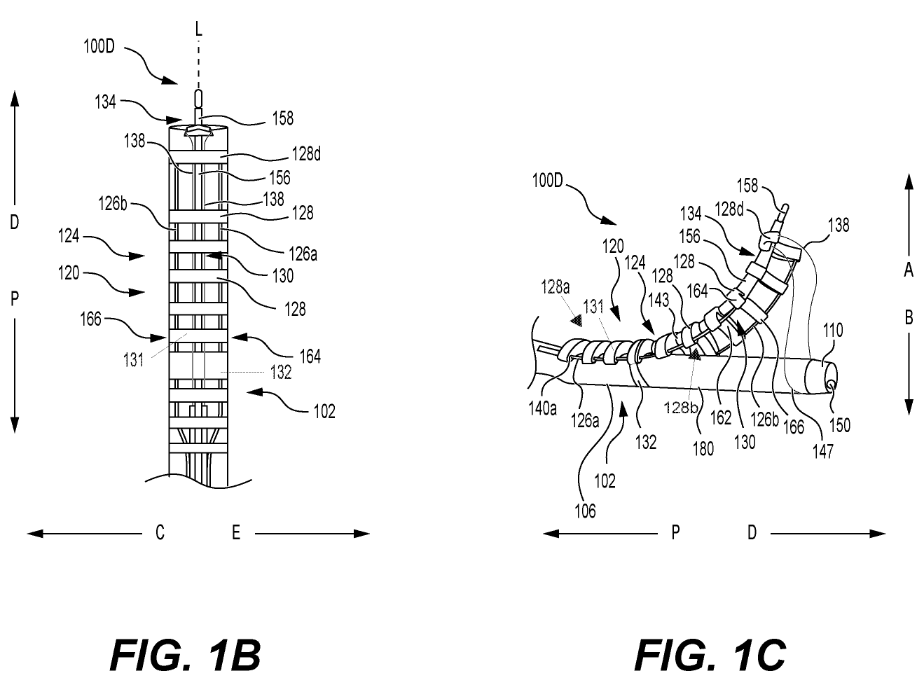

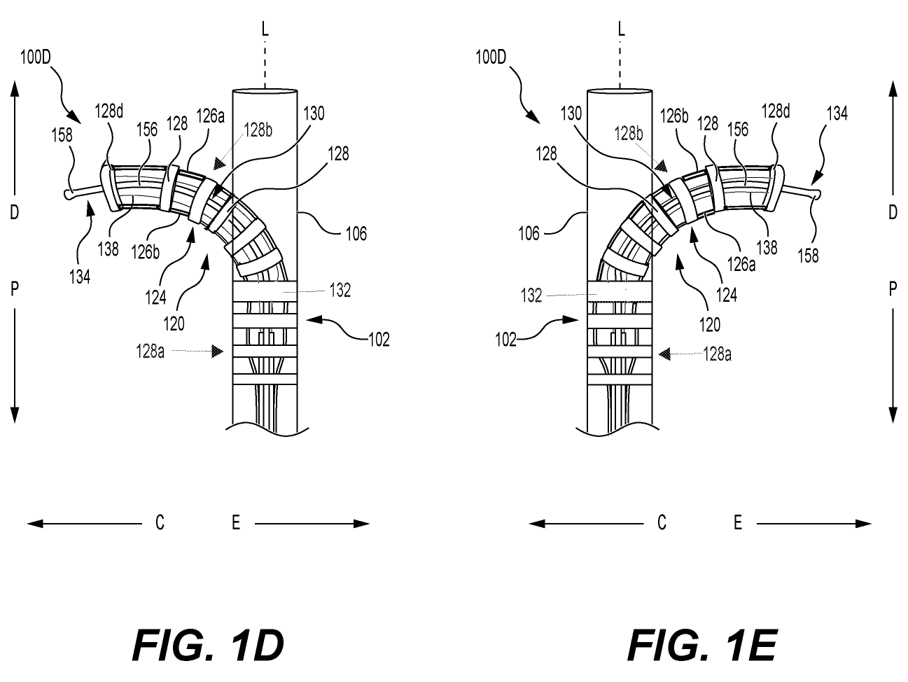

FIG. 1A illustrates an exemplary medical system 100, and FIGS. 1B-1E illustrate various views of a distal portion 100D of medical system 100. Medical system 100 may include a first medical device 102 (e.g., an endoscope), a second medical device 120 (e.g., an accessory device), and a sheath 142 defining a lumen 168. First medical device 102 and second medical device 120 may be received within lumen 168 of sheath 142, and distal portions of first medical device 102 and second medical device 120 may extend distally of a distal end of sheath 142. As discussed in detail below, distal portions of first medical device 102 and second medical device 120 may both be articulable (e.g., separately articulable).

First medical device 102 may include a handle 104 and a shaft 106, with shaft 106 extending distally from handle 104. Shaft 106 may include an articulation section 180 and a distal tip 110. Distal tip 110 may include one or more imaging devices (e.g., a camera, an imager, etc.), one or more light sources (e.g., light emitting diode(s), optical fibers, etc.), and a distal opening in communication with a working channel 150 extending through shaft 106 (shown in FIG. 1C).

Handle 104 may include a port 118 in fluid communication with working channel 150. Handle 104 may include a first actuator 112 and a second actuator 114 (e.g., knobs or levers) for controlling articulation section 180. For example, actuators 112, 114 may control movement of articulation section 180 and distal tip 110 in multiple directions, for example, movement along different planes. Handle 104 may be coupled to an umbilicus 116. Umbilicus 116 may extend from handle 104 to capital equipment, such as electronic controller(s), and/or source(s) of air, water, and/or suction.

Second medical device 120 may extend distally alongside at least a portion of first medical device 102. Second medical device 120 includes a proximal portion 122 and a distal portion 124. Proximal portion 122 may include a handle 136, and distal portion 124 may include a channel 130 (e.g., an external channel). A section of distal portion 124 may be fixed to shaft 106, for example, at a position on shaft 106 proximate and/or proximal to articulation section 180 via at least one attachment device 132 (e.g., a strap, a clip, a band, or other attachment device).

Distal portion 124 may include a plurality of ribs 128, and each of the plurality of ribs 128 may include a rib body 131 extending from a first lateral end 164 to a second lateral end 166 (FIG. 1B). Each rib body 131 may be substantially arc-shaped or curved from first lateral end 164 to second lateral end 166, for example, such that each rib body 131 may follow a contour of a portion of shaft 106. For example, as better shown in FIG. 1C, each rib body 131 may include a curved surface 162 complementary to an outermost surface of shaft 106. Each rib body 131 may include a plurality of openings that extend longitudinally through portions of rib body 131. For example, each rib body 131 may include a first opening 140a disposed proximate first lateral end 164, a second opening 140b disposed proximate second lateral end 166, and a central opening 143. Channel 130 may extend through rib bodies 131 of the plurality of ribs 128, and channel 130 may be partially defined by central openings 143 of rib bodies 131. In some examples, channel 130 may be defined by a tube (e.g., a flexible tube) extending through central openings 143 of rib bodies 131.

Channel 130 may be configured to receive a medical instrument 134, including a shaft 156 and an end effector 158 (e.g., an electrode, forceps, a biopsy device, etc.) coupled to a distal end of shaft 156. Shaft 156 of medical instrument 134 may be movable (e.g., longitudinally) within channel 130. First openings 140a may be configured to receive a first cable 126a, and second openings 140b may be configured to receive a second cable 126b. First cable 126a and second cable 126b may be movable for articulating (e.g., bending) or otherwise moving distal portion 124 (and thus channel 130) relative to a longitudinal axis L of shaft 106, which will be discussed in greater detail below.

The plurality of ribs 128 may include a first set of ribs 128a (e.g., proximal ribs) and a second set of ribs 128b (e.g., distal ribs) distal to attachment device 132. Alternatively, all of the ribs 128 may be distal to attachment device 132. In another alternative, attachment device 132 may be omitted and sheath 142 may be of a suitable size and shape to maintain a section of distal portion 124 fixed to or otherwise adjacent to shaft 106, while allowing a remaining section of distal portion 124 to articulate or move relative to longitudinal axis L of shaft 106. A section of distal portion 124 being fixed at a position proximate and/or proximal to articulation section 180 may allow for distal portion 124 and articulation section 180 (and distal tip 110) to be articulated or moved independent of one another. It will be appreciated that although ten ribs 128 are illustrated in FIGS. 1A-1E, distal portion 124 of second medical device 120 may include any number of ribs 128 and that the plurality of ribs 128 may be evenly or unevenly spaced apart. It will also be appreciated that although one attachment device 132 is illustrated in FIGS. 1A-1E, medical system 100 may include more than one attachment device 132.

First and second cables 126a, 126b may extend distally from handle 136, through sheath 142, and through the plurality of ribs 128 via their respective openings 140a, 140b. Distal ends of first and second cables 126a, 126b may be fixed to a distalmost rib 128d. For example, distal ends of first and second cables 126a, 126b may be fixed within their respective openings 140a, 140b of distalmost rib 128d, while the remaining portions of first and second cables 126a, 126b may move longitudinally within their respective openings 140a, 140b of the remaining ribs 128. Proximal ends of first and second cables 126a, 126b may be coupled to one or more actuators of handle 136 (e.g., a knob, a slider, a joystick, a lever). The one or more actuators of handle 136 may be configured to actuate first and second cables 126a, 126b to articulate or move distal portion 124 relative to longitudinal axis L of shaft 106.

FIGS. 1A and 1B illustrate distal portion 124 in a first configuration or a neutral configuration in which distal portion 124 may be coupled to shaft 106. In the neutral configuration, the plurality of ribs 128 may contact shaft 106 and distal portion 124 (and thus channel 130) may be aligned with longitudinal axis L of shaft 106. In the neutral configuration, movement of distal portion 124 of second medical device 120 may be coupled to movement of shaft 106 of first medical device 102. For example, in the neutral configuration, distal portion 124 of second medical device 120 may articulate or move with shaft 106 of first medical device 102.

FIGS. 1C-1E illustrate distal portion 124 in articulated configurations, in which distal portion 124 may be at a non-zero angle relative to longitudinal axis L of shaft 106. In the articulated configurations (e.g., bent configurations), one or more distal ribs 128b may be separated or decoupled from shaft 106 as shown in FIGS. 1C-1E. As shown in FIG. 1C, first and second cables 126a, 126b may be pulled or otherwise urged proximally (e.g., tension may be applied) simultaneously to articulate distal portion 124 in an A direction (e.g., an up direction) relative to longitudinal axis L of shaft 106. As shown in FIG. 1D, second cable 126b may be pulled or otherwise urged proximally to articulate distal portion 124 in a C direction (e.g., a left direction) relative to longitudinal axis L of shaft 106. As shown in FIG. 1E, first cable 126a may be pulled or otherwise urged proximally to articulate distal portion 124 in an E direction (e.g., a right direction) relative to longitudinal axis L of shaft 106. To return distal portion 124 to the neutral configuration, tension may be removed from first and second cables 126a, 126b. For example, to transition the distal portion 124 from the articulated configuration illustrated in FIG. 1C to the neutral configuration illustrated in FIGS. 1A and 1B, first and second cables 126a, 126b may be moved distally simultaneously to thereby move distal portion 124 towards shaft 106 in a B direction (e.g., a down direction).

In some examples, handle 136 may include one or more springs or biasing mechanisms to help return distal portion 124 to the neutral configuration. During a procedure, an operator may control proximal and distal movement of first and second cables 126a, 126b to articulate and/or angle distal portion 124 relative to longitudinal axis L of shaft 106 in a number of different directions.

As discussed above, medical instrument 134 may be received within channel 130 of distal portion 124. A distal portion of shaft 156 may be received within channel 130 of distal portion 124, such that articulation or movement of distal portion 124 in a direction, articulates or moves the distal portion of shaft 156 and end effector 158 in the same direction. The distal portion of shaft 156 may be formed of a flexible material, which may help to allow the distal portion of shaft 156 to articulate or move with distal portion 124 of second medical device. In some examples, distal portion 124 of second medical device 120 may include one or more imaging devices (e.g., a camera) and/or one or more light sources, which may help with visualization of a target site and/or medical instrument 134 during a procedure. For example, the one or more imaging devices and the one or more light sources may be disposed at a distal end of distal portion 124.

Medical system 100 may further include a return mechanism, such as a thread 138 (e.g., a string, a wire), tethering distal portion 124 to shaft 106. Thread 138 may be looped, wrapped, or otherwise secured around or to distal tip 110 of shaft 106. Additionally, another portion of thread 138 may extend proximally along distal portion 124, through sheath 142, and to proximal portion 122 of second medical device 120. A looped portion 147 of thread 138 may be attached to distal tip 110. Thread 138 (e.g., ends of thread 138) may be coupled to a control mechanism on handle 136 that may allow for thread 138 to loosen or slacken when distal portion 124 is articulated or moved relative to shaft 106 (as better shown in FIG. 1C). The control mechanism may also allow for tension to be applied to thread 138 to draw shaft 106 towards distal portion 124 of second medical device 120. For example, during insertion of medical system 100 into a body lumen or removal of medical system 100 from a body lumen, tension may be applied to thread 138 to help secure distal portion 124 to shaft 106, which may help to ensure safe insertion or removal. In some examples, tension may be applied to thread 138 to help secure distal portion 124 of second medical device 120 to shaft 106 of first medical device 102 to help with visualization. For example, the one or more imaging devices and the one or more light sources at distal tip 110 of shaft 106 may help with visualization of a target site, distal portion 124 of second medical device 120 and/or medical instrument 134, for example, when distal portion 124 and/or medical instrument 134 extends distally beyond distal tip 110 of shaft 106.

Alternatively, thread 138 may be looped, wrapped, or otherwise secured around or to distal portion 124 of second medical device 120, and another portion of thread 138 may extend proximally through working channel 150 of shaft 106. In this alternative, looped portion 147 of thread 138 may be attached to distal portion 124 of second medical device 120 (e.g., attached to distalmost rib 128d or other rib 128), such that when tension is applied to thread 138, distal portion 124 of second medical device 120 may be drawn or otherwise urged towards shaft 106. In yet another alternative, thread 138 may be omitted, for example, when distal portion 124 of second medical device 120 includes the one or more imaging devices.

Although only one second medical device 120 is illustrated in FIGS. 1A-1E, it will be appreciated that medical system 100 may include any number of second medical devices 120 coupled to first medical device 102. For example, medical system 100 may include two second medical devices 120 with their respective distal portions 124 disposed on opposite sides of shaft 106 for delivering multiple medical instruments to a target site. In other aspects, medical system 100 may include three or more second medical devices 120, for example, evenly or unevenly spaced around and coupled to shaft 106.

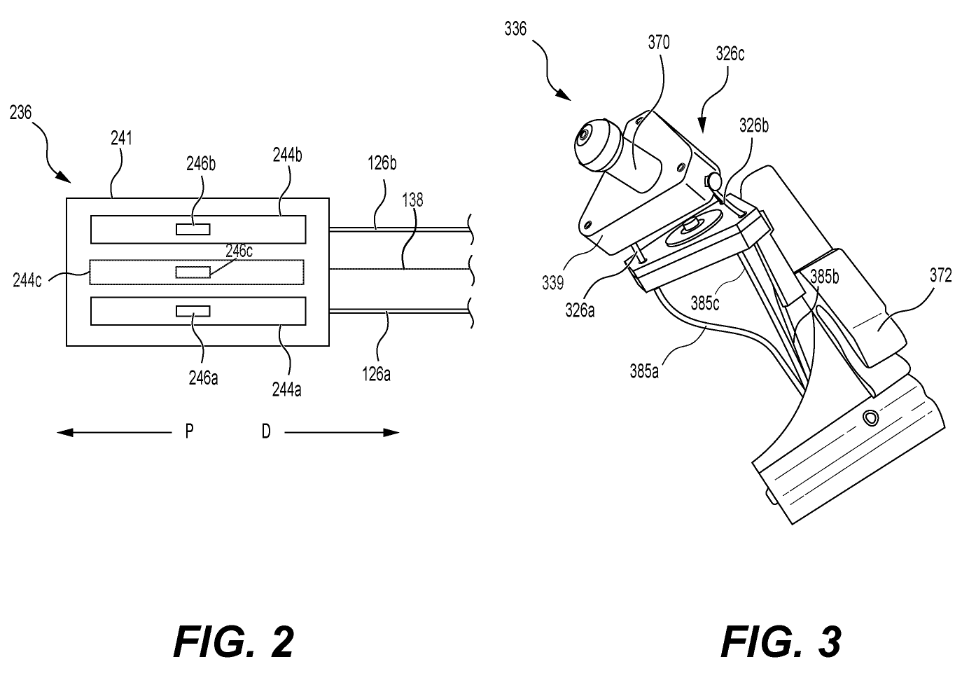

FIG. 2 illustrates a schematic view of an exemplary handle 236 that may be used in place of handle 136 for controlling movement of first and second cables 126a, 126b to articulate or move distal portion 124 relative to longitudinal axis L of shaft 106, and for controlling movement of thread 138. Handle 236 may include a housing 241 having a first channel 244a, a second channel 244b, and a third channel 244c. Channels 244a, 244b, 244c may extend longitudinally along respective portions of housing 241.

Handle 236 may include a first slider 246a movable within first channel 244a, and handle 236 may also include a second slider 246b movable within second channel 244b. First slider 246a may be coupled to the proximal end of first cable 126a, and second slider 246b may be coupled to the proximal end of second cable 126b. First and second sliders 246a, 246b may be moved proximally or distally within their respective channels 244a, 244b to correspondingly move their respective first and second cables 126a, 126b proximally or distally. Handle 236 may further include a third slider 246c movable within third channel 244c, and third slider 246c may be coupled to thread 138. Slider 246c may be moved proximally or distally within channel 244c to correspondingly move thread 138 proximally or distally.

In some examples, although not shown, handle 236 may include one or more locking mechanisms to help maintain a position of sliders 246a, 246b, 246c within their respective channels 244a, 244b, 244c. For example, the one or more locking mechanisms may help maintain a position of first and second sliders 246a, 246b to thereby maintain a position of their respective first and second cables 126a, 126b, and thus maintain a position of distal portion 124 of second medical device 120.

Alternatively, three cables may be used to articulate or move distal portion 124 relative to shaft 106. For example, first and second cables 126a, 126b may be used to articulate or move distal portion 124 relative to longitudinal axis L of shaft 106 in the C and E directions (e.g., left and right directions), and a third cable may be used to articulate or move distal portion 124 relative to longitudinal axis L of shaft 106 in the A and B directions (e.g., up and down directions). In this example, an exemplary handle 336 illustrated in FIG. 3 may be used to actuate three cables. Handle 336 may be a joystick-type handle that includes an actuator 370 coupled to a first cable 326a, a second cable 326b, and a third cable 326c. Handle 336 may include a base 339. Cables 326a, 326b, 326c may extend through respective openings in base 339 and through respective sheaths or tubes (e.g., hypotubes) 385a, 385b, 385c. Tubes 385a, 385b, 385c may be rigid, for example, formed of a metal. Cables 326a, 326b, 326c may extend distally from handle 336 to a distalmost rib 128 of distal portion 124. In this example, ribs 128 of distal portion 124 may each include three equally spaced openings for receiving cables 326a, 326b, 326c.

Actuator 370 may be movable in multiple directions to articulate or move distal portion 124 of second medical device in the up (direction A), down (direction B), left (direction C), and right (direction E) directions relative to longitudinal axis L of shaft 106. Handle 336 may include a coupling mechanism 372 for attaching handle 336 to first medical device 102. For example, coupling mechanism 372 may include a clip, a clamp, a strap, a band, or other coupling mechanism.

In another alternative, first and second cables 326a, 326b may be coupled to a knob for controlling articulation or movement of distal portion 124 relative to longitudinal axis L of shaft 106 in the C and E directions (e.g., left and right directions), and third cable 326c may be coupled to a slider for controlling articulation or movement of distal portion 124 relative to longitudinal axis L of shaft 106 in the A and B directions (e.g., up and down directions).

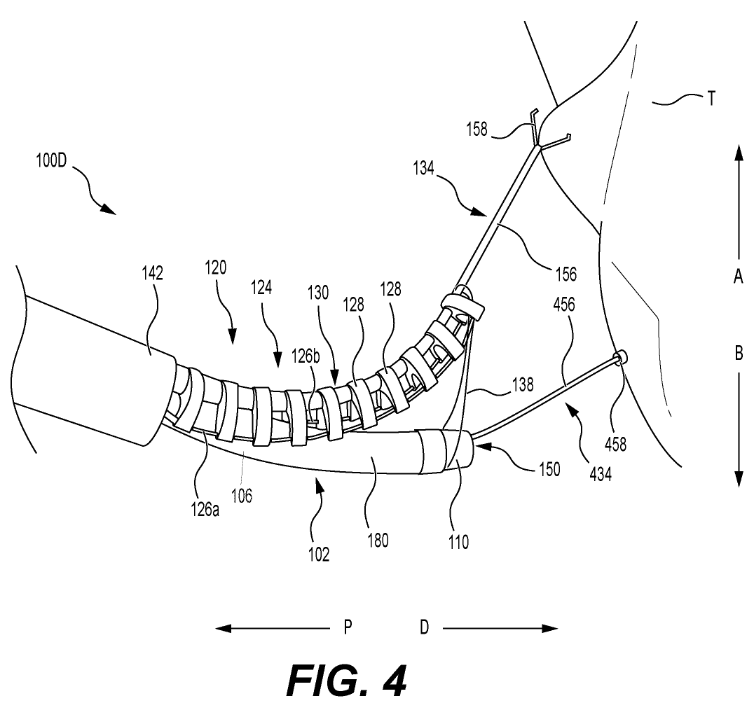

A method of using medical system 100 will now be described with reference to FIG. 4, which illustrates distal portion 100D of medical system 100 at a target site. Distal portion 100D of medical system 100 may be advanced to a target site in a body lumen (e.g., the gastrointestinal tract) with distal portion 124 of second medical device 120 in the neutral configuration (illustrated in FIGS. 1A and 1B). For example, distal portion 124 of second medical device 120 may be coupled to shaft 106 during advancement of medical system 100 to the target site.

Once distal portion 100D of medical system 100 is proximate the target site, an operator may decouple distal portion 124 of second medical device 120 from shaft 106 by actuating the one or more actuators on handle 136 to articulate distal portion 124 (and thus channel 130) of second medical device 120 relative to longitudinal axis L of shaft 106. In this example, the operator may articulate distal portion 124 of second medical device 120, and thus the distal portion of shaft 156 of medical instrument 134, in the A direction (e.g., up direction) by pulling first and second cables 126a, 126b proximally simultaneously. The operator may continue to move or articulate distal portion 124 of second medical device 120 relative to longitudinal axis L of shaft 106 by manipulating first and second cables 126a, 126b until distal portion 124 of second medical device 120 is in a desired position.

Once distal portion 124 of second medical device 120 is in a desired position, the operator may advance shaft 156 of medical instrument 134 distally through channel 130 to position end effector 158 at tissue T at the target site. For example, medical instrument 134 may be positioned within channel 130 such that a distal end of medical instrument 134 is retracted within channel 130 during delivery to the target site, and then medical instrument 134 may be advanced distally once distal portion 124 of second medical device 120 is proximate the target site and/or in a desired position. In this example, end effector 158 may be forceps. Once end effector 158 is in a desired position, the operator may actuate or otherwise manipulate end effector 158 to grasp tissue T at the target site. Additionally, medical instrument 134, including end effector 158, may be moved or urged in one or more directions (e.g., proximally via movement of medical instrument 134, in one or more directions via movement of second medical device 120, etc.) to help apply traction and/or tension to tissue T.

While the desired position of distal portion 124 of second medical device 120 and end effector 158 is maintained, the operator may articulate articulation section 180 and distal tip 110 of shaft 106 relative to and independent of distal portion 124 of second medical device 120 to a desired position using actuators 112, 114 on handle 104. The operator may then advance a second medical instrument 434 through working channel 150 of shaft 106 to the target site. Second medical instrument 434 may include a shaft 456 and an end effector 458 (e.g., an electrode, a biopsy device, etc.) coupled to a distal end of shaft 456. The operator may use end effector 458 to cut, ablate, or otherwise treat tissue T at the target site, while tissue traction is maintained by end effector 158. In some examples, end effector 158 may be used to hold onto tissue T to be removed from the target site, or released to be passed or otherwise removed.

The ability of shaft 106 of first medical device 102 and distal portion 124 of second medical device 120 to articulate or move independently of one another may allow for the operator to articulate shaft 106 of first medical device 102 to treat tissue T without changing the traction vector or otherwise repositioning second medical device 120. In some examples, during use, the operator may articulate or move distal portion 124 of second medical device 120 and articulation section 180 (and distal tip 110) of shaft 106 in different directions independently of one another to reposition end effectors 158, 458 relative to tissue T.

To remove medical system 100 from the body lumen, the operator may retract medical instrument 134 into channel 130 and may retract second medical instrument 434 into working channel 150. The operator may then remove tension from first and second cables 126a, 126b to return distal portion 124 of the second medical device 120 to the neutral configuration. Alternatively or additionally, the operator may apply tension to thread 138 to secure distal portion 124 of second medical device 120 to shaft 106 of first medical device 102 before withdrawing medical system 100 from the body lumen.

In an alternative example, distal portion 100D of medical system 100 may be advanced to the target site with distal portion 124 of second medical device 120 in the neutral configuration. Once distal portion 100D of medical system 100 is proximate the target site, the operator may articulate articulation section 180 and distal tip 110 of shaft 106 to position distal tip 110 at a desired position. The operator may then advance second medical instrument 434 through working channel 150 of shaft 106 to the target site. In this example, end effector 458 may be forceps. The operator may actuate or otherwise manipulate end effector 458 to grasp tissue T.

While the desired position of distal tip 110 of shaft 106 and end effector 458 is maintained, the operator may articulate distal portion 124 of second medical device 120 relative to longitudinal axis L of shaft 106. Once distal portion 124 of second medical device 120 is in a desired position, the operator may advance shaft 156 of medical instrument 134 distally through channel 130 to position end effector 158 at the target site. In this example, the operator may use end effector 158 to cut, ablate, or otherwise treat tissue T at the target site, while tissue traction is maintained by end effector 458.

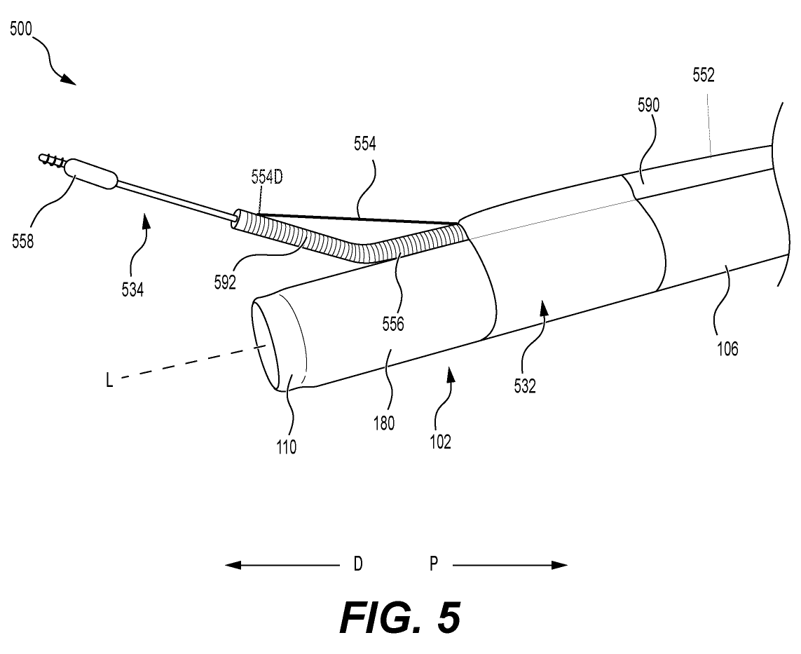

FIG. 5 illustrates another exemplary medical system 500 including first medical device 102 and a sheath 552 extending distally alongside shaft 106 of first medical device 102. Sheath 552 may be coupled to shaft 106 via at least one attachment device 532 (e.g., a clip, a strap, a band, or other attachment device). Attachment device 532 may help to fix sheath 552 to shaft 106 at a position proximate and/or proximal to articulation section 180. Sheath 552 may define a lumen 590 configured to receive a medical instrument 534 having any of the features of medical instruments 134, 434. For example, medical instrument 534 may include a shaft 556 and an end effector 558 at a distal end of shaft 556.

In this example, shaft 556 may include a distal portion 592, and distal portion 592 may be bent or movable relative to the remaining portions of shaft 556 and/or relative to longitudinal axis L of shaft 106. Distal portion 592 of shaft 556 may be formed of a flexible material, and the remaining portions of shaft 556 may be formed of a rigid or semi-rigid material. For example, distal portion 592 of shaft 556 may be a coil. Distal portion 592 of shaft 556 may be shorter than the remaining portions of shaft 556. Distal portion 592 may be movable between a neutral configuration and an articulated (e.g., bent) configuration. In the neutral configuration, distal portion 592 and end effector 558 (e.g., a grasper, a helix, etc.) may be aligned with longitudinal axis L of shaft 106 and may contact the outermost surface of shaft 106. As shown in FIG. 5, in the articulated configuration, distal portion 592 and end effector 558 may be at a non-zero angle relative to longitudinal axis L of shaft 106.

Medical system 500 may further include a control wire 554 extending from a proximal end to a distal end. Distal end 554D of control wire 554 may fixed to distal portion 592. Control wire 554 may extend proximally through lumen 590 of sheath 552. The proximal end (not shown) of control wire 554 may be coupled to an actuator for controlling actuation of control wire 554 to move distal portion 592 between the neutral and articulated configurations. For example, proximal movement of control wire 554 may move distal portion 592 and end effector 558 away from shaft 106 toward the articulated configuration, and distal movement of control wire 554 may move distal portion 592 and end effector 558 towards shaft 106 toward the neutral configuration. As shown in FIG. 5, due to attachment device 532 being positioned proximate and/or proximal to articulation section 180, distal portion 592 and articulation section 180 (and distal tip 110) may be articulated or moved independent of one another.

Although only one sheath 552 and only one medical instrument 534 is illustrated in FIG. 5, it will be appreciated that medical system 500 may include more than one sheath 552 and more than one medical instrument 534 coupled to shaft 106. In some examples, sheath 552 may define more than one lumen 590 for receiving multiple medical instruments 534.

It will be apparent to those skilled in the art at various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

What is claimed is:

1. A medical system comprising:
a first medical device comprising:
a handle and a shaft extending distally from the handle; and
a second medical device coupled to at least a portion of the first medical device, the second medical device comprising:
a distal portion including a plurality of ribs and a channel, wherein the channel extends through the plurality of ribs; and
a plurality of cables coupled to the plurality of ribs, wherein each cable of the plurality of cables is configured to move the distal portion of the second medical device from a first configuration to a second configuration, wherein, in the first configuration, the distal portion of the second medical device is aligned with a longitudinal axis of the shaft of the first medical device, and wherein, in the second configuration, the distal portion of the second medical device is at a non-zero angle relative to the longitudinal axis of the shaft of the first medical device.

2. The medical system of claim 1, wherein a distal end of each of the plurality of cables is fixed to a distalmost rib of the plurality of ribs.

3. The medical system of claim 1, wherein each of the plurality of ribs includes two openings that extend longitudinally through portions of each of the plurality of ribs, and wherein the two openings are configured to receive the plurality of cables.

4. The medical system of claim 3, wherein each of the plurality of ribs includes a central opening that partially defines the channel.

5. The medical system of claim 4, wherein each of the plurality of ribs contacts the shaft of the first medical device in the first configuration, and wherein one or more distal ribs of the plurality of ribs is separated from the shaft of the first medical device in the second configuration.

6. The medical system of claim 1, wherein each of the plurality of ribs includes a curved surface complementary to an outermost surface of the shaft of the first medical device.

7. The medical system of claim 1, wherein a section of the distal portion of the second medical device is coupled to the shaft of the first medical device via an attachment device.

8. The medical system of claim 1, further comprising a return mechanism configured to move the distal portion of the second medical device towards the shaft of the first medical device.

9. The medical system of claim 8, wherein the return mechanism comprises a thread, and wherein the thread wraps around the shaft of the first medical device.

10. The medical system of claim 1, wherein the channel of the second medical device is configured to receive a medical instrument.

11. The medical system of claim 10, wherein, when the medical instrument is received within the channel of the second medical device, movement of the distal portion of the second medical device correspondingly moves the medical instrument.

12. The medical system of claim 1, wherein the plurality of cables includes a first cable and a second cable, wherein the first cable extends through a portion of each of the plurality of ribs proximate a first lateral end of each of the plurality of ribs, and wherein the second cable extends through a portion of each of the plurality of ribs proximate a second lateral end of each of the plurality of ribs.

13. The medical system of claim 1, wherein the second medical device comprises a handle comprising at least one actuator coupled to the plurality of cables, and wherein the at least one actuator is configured to move the plurality of cables proximally or distally to move the distal portion of the second medical device between the first configuration and the second configuration.

14. The medical system of claim 13, wherein the at least one actuator includes a slider, a joystick, or a knob.

15. The medical system of claim 1, further comprising a sheath defining a lumen, wherein the lumen is configured to receive at least the distal portion of the second medical device and the shaft of the first medical device.

16. A medical system comprising:
a first medical device comprising:
a handle; and
a shaft extending distally from the handle, wherein the shaft includes a longitudinal axis, an articulation section, and a distal tip; and
a second medical device extending distally alongside at least a portion of the shaft of the first medical device, the second medical device comprising:
a distal portion including a channel;
a first cable; and
a second cable,
wherein the first cable and the second cable are configured to move proximally or distally to move the channel between a first configuration and a second configuration, and wherein, in the first configuration, the channel of the second medical device is aligned with the longitudinal axis of the shaft of the first medical device, and wherein, in the second configuration, the channel of the second medical device is at a non-zero angle relative to the longitudinal axis of the shaft of the first medical device.

17. The medical system of claim 16, wherein the distal portion includes a plurality of ribs, and wherein the channel extends through the plurality of ribs.

18. The medical system of claim 16, wherein a section of the distal portion is fixed to the shaft of the first medical device at a position on the shaft proximal to the articulation section.

19. A method of treating a target site:
delivering a medical system to the target site, the medical system comprising a first medical device and a second medical device, wherein the second medical device is coupled to at least a portion of the first medical device;
articulating a distal portion of the first medical device to a first position;
advancing a first medical instrument through a working channel of the first medical device;
articulating a distal portion of the second medical device to a second position independent of the distal portion of the first medical device; and
advancing a second medical instrument through a channel of the second medical device.

20. The method of claim 19, wherein the distal portion of the second medical device includes a plurality of ribs, and wherein the channel extends through the plurality of ribs.
